(19) United States
(12) Patent Application Publication
(10) Pub. No.: US 2013/0297340 A1
(43) Pub. Date: Nov. 7, 2013

(54) LEARNING AND OPTIMIZING CARE PROTOCOLS

(71) Applicants: Cornelis Conradus Adrianus Maria Van Zon, Fishkill, NY (US); Charles Lagor, Ardsley, NY (US); William Lord, Fishkill, NY (US); Stephen Hermann Rudolf Theiss, Duesseldorf (DE)

(72) Inventors: Cornelis Conradus Adrianus Maria Van Zon, Fishkill, NY (US); Charles Lagor, Ardsley, NY (US); William Lord, Fishkill, NY (US); Stephen Hermann Rudolf Theiss, Duesseldorf (DE)

(73) Assignee: KONINKLIJKE PHILIPS ELECTRONICS N.V., Eindhoven (NL)

(21) Appl. No.: 13/996,175

(22) PCT Filed: Dec. 9, 2011
(86) PCT No.: PCT/IB2011/055571

§ 371 Date: Jun. 20, 2013

Related U.S. Application Data
(60) Provisional application No. 61/425,394, filed on Dec. 21, 2010.

(51) Int. Cl. G06Q 10/06; G06Q 50/22

(52) U.S. Cl. CPC G06Q 10/0633; G06Q 50/22
USPC 705/2

(57) ABSTRACT

A method (600) of learning and/or optimizing clinical protocols and/or clinical guidelines. Workflow data is received (602) for a plurality of patients. The workflow data includes a plurality of care steps and relations there between for each of the patients. One or more sequences of care steps are identified (604) from the workflow data. The identification includes determining (612) one or more most common sequences of care steps from the workflow data. The identified sequences of care steps include the most common sequences of care steps. One of the identified sequences of care steps is selected (606) and employed (608) as a clinical protocol and/or to generate a clinical guideline.

The present application relates generally to clinical decision making It finds particular application in conjunction with learning and/or optimizing clinical protocols and/or clinical guidelines, and will be described with particular reference thereto. However, it is to be understood that it also finds application in other usage scenarios, and is not necessarily limited to the aforementioned application.

Clinical protocols (also referred to as care protocols) are the procedures established by medical institutions, such as hospitals, to follow when caring for patients. These protocols aim at ensuring the health and safety of patients while minimizing the cost of care. Typically, clinical protocols are derived from clinical guidelines. Clinical guidelines are recommendations on the appropriate treatment and care for people with specific diseases and conditions based on the best available evidence. Further, they are typically independent of any specific medical institution. Expert bodies, such as the American Heart Association, the American Diabetes Association, the Institute for Clinical Systems Improvement, and the like, publish these clinical guidelines.

One challenge medical institutions often face is a lack of explicit protocols for managing every disease or condition. Without clinical protocols to follow, clinicians, such as doctors, nurses, healthcare professionals, healthcare assistants, and the like, generally rely upon experience. However, as is to be appreciated, this leads to varying treatment approaches from clinician to clinician, which negatively impacts the standard of care and/or cost of care for patients and/or medical institutions.

Regardless of this challenge, a significant trend in healthcare has been an increasing expectation for medical institutions to adhere to clinical guidelines. Clinical guidelines are increasingly viewed as sources of “best-practice” standards of care. Further, adherence to the recommendations of clinical guidelines has been shown to reduce costs and improve outcomes, whereby performance measures and reimbursements are increasingly tied to guideline adherence in at least the United States. Accordingly, there is a growing need for guideline-based clinical decision support systems (CDSSs) to support medical institutions in adhering to clinical guidelines.

A guideline-based CDSS is a system that provides one or more of administrators, clinicians, patients, and the like with clinical recommendations that are intelligently filtered, patient specific, and presented at the point of care at appropriate times. To accomplish this, a guideline-based CDSS executes computer interpretable guidelines (CIGs) on a per patient basis. A CIG typically embodies a clinical protocol of the medical institution employing the CIG, and therefore is typically comprised of a plurality of care steps and incorporates recommendations as to how to treat and/or care for a patient based on the present care step. Therefore, CIGs embodying clinical protocols derived from clinical guidelines support medical institutions in adhering to the clinical guidelines.

The present application provides a new and improved systems and methods of learning and/or optimizing clinical protocols and/or clinical guidelines which overcomes the above-referenced problems and others.

In accordance with one aspect, a method of learning and/or optimizing clinical protocols and/or clinical guidelines is provided. Workflow data is received for a plurality of patients. The workflow data includes a plurality of care steps and relations therebetween for each of the patients. One or more sequences of care steps are identified from the workflow data. The identification includes determining one or more most common sequences of care steps from the workflow data. The identified sequences of care steps include the most common sequences of care steps. One of the identified sequences of care steps is selected. One or more care steps of the selected sequence of care steps are then integrated into an established clinical protocol and/or the selected sequence of care steps is employed as a clinical protocol and/or to provide feedback to expert bodies that generate clinical guidelines.

In accordance with another aspect, a system for learning and/or optimizing clinical protocols and/or clinical guidelines is provided. The system includes a data collection engine that receives workflow data for a plurality of patients. The workflow data includes a plurality of care steps and relations therebetween for each of the patients. The system further includes a data analysis engine that identifies one or more care steps from the workflow data. The identification includes determining one or more most common sequences of care steps from the workflow data. The identified care steps include the care steps of the most common sequences of care steps. The data analysis engine is further operative to select one or more of the identified care steps. Even more, the data analysis engine is operative to integrate the selected care steps into an established clinical protocol and/or employ the selected care steps as a clinical protocol and/or to provide feedback to expert bodies that generate clinical guidelines.

In accordance with another aspect, a medical system is provided. The medical system includes one or more sources of workflow data and a guideline-based clinical decision support system (CDSS). The guideline-based CDSS includes one or more processors programmed to receive workflow data for a plurality of patients from the sources. The workflow data includes a plurality of care steps and relations therebetween for each of the patients. The one or more processors are further programmed to identify one or more sequences of care steps from the workflow data. The identification includes determining one or more common sequences of care steps from the workflow data. The one or more processors are further programmed to select one of the identified sequences of care steps. Even more, the processors are programmed to integrate one or more care steps of the selected sequence of care steps into an established clinical protocol and/or to employ the selected sequence of care steps as a clinical protocol and/or to provide feedback to expert bodies that generate clinical guidelines.

One advantage of the present systems and methods resides in the ability learn and/or optimize clinical protocols and/or facilitate the generation of clinical guidelines.

Another advantage resides in the ability to learn clinical protocols and/or clinical guidelines known by clinicians but not explicitly documented and/or evaluated.

Another advantage resides in the ability to learn clinical protocols and/or clinical guidelines for different subpopulations of patients.

Another advantage resides in the ability to evaluate clinical protocols and/or clinical guidelines based on performance.

Another advantage resides in the ability to update established clinical protocols and/or clinical guidelines.

Still further advantages of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

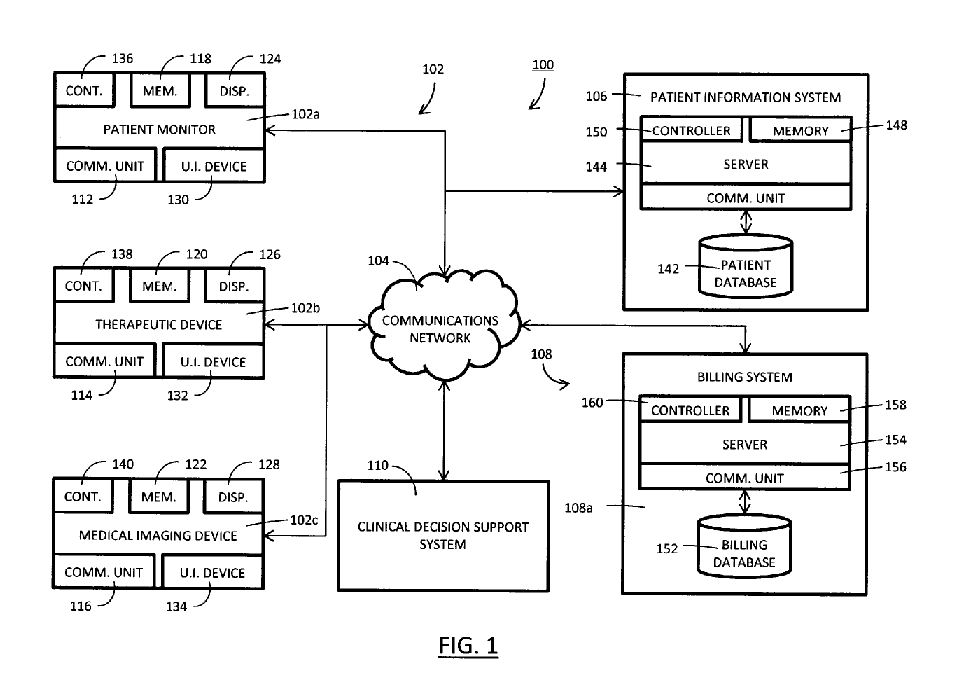

FIG. 1 is a block diagram an information technology (IT) infrastructure of a medical institution according to aspects of the present disclosure;

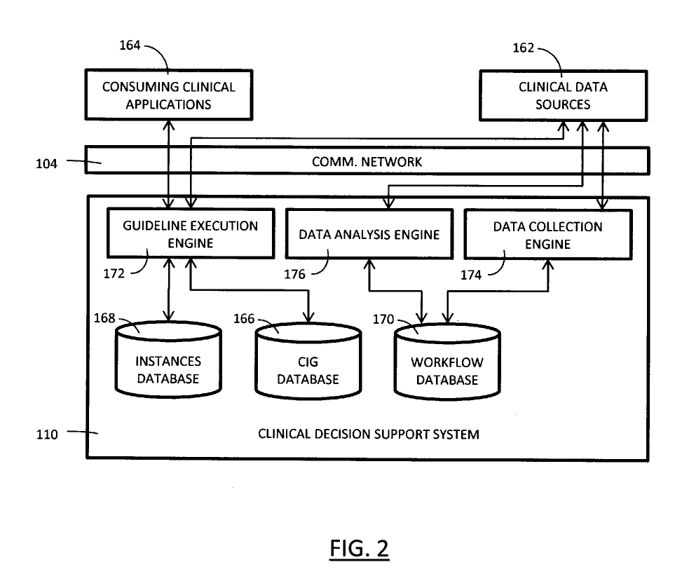

FIG. 2 is a block diagram of functional components of a guideline-based clinical decision support system (CDSS) according to aspects of the present disclosure;

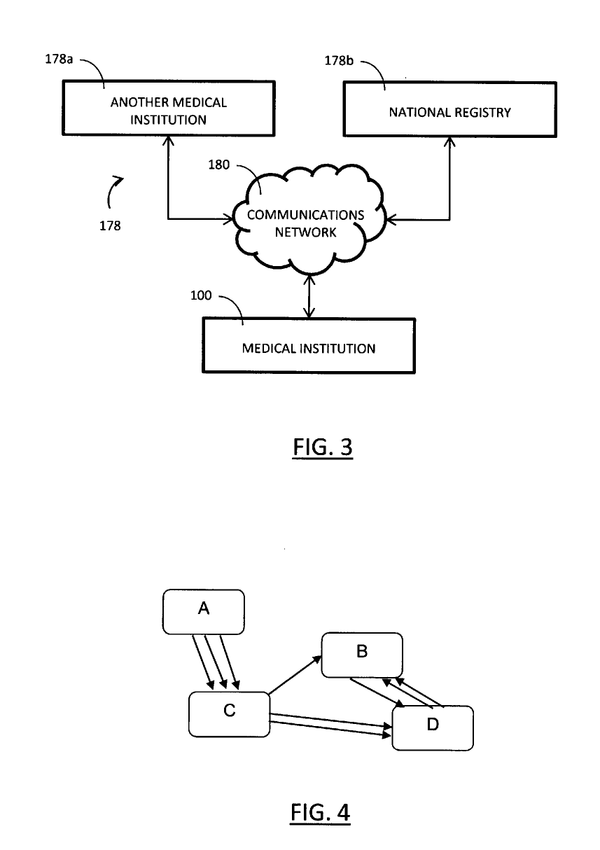

FIG. 3 is a block diagram of external sources from which workflow data can be collected;

FIG. 4 is a graphical illustration of workflow data;

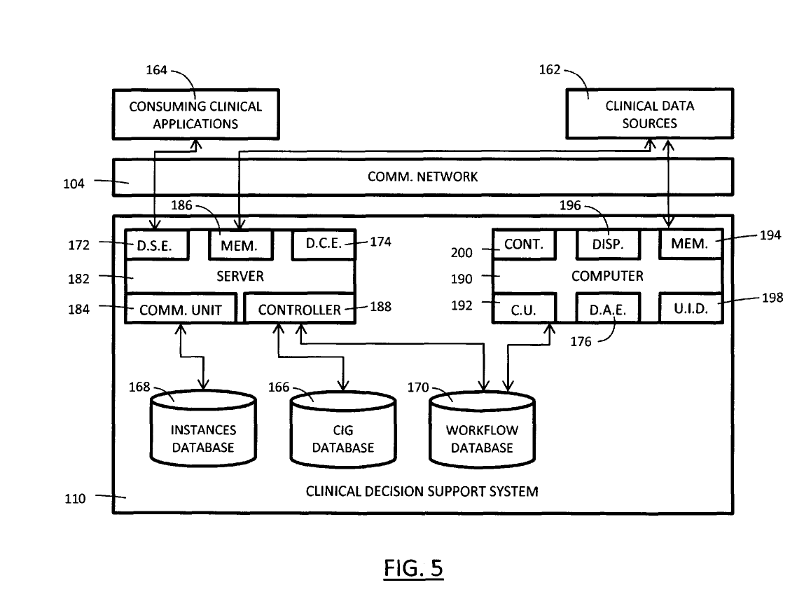

FIG. 5 is a block diagram of structural components of a guideline-based CDSS according to aspects of the present disclosure; and,

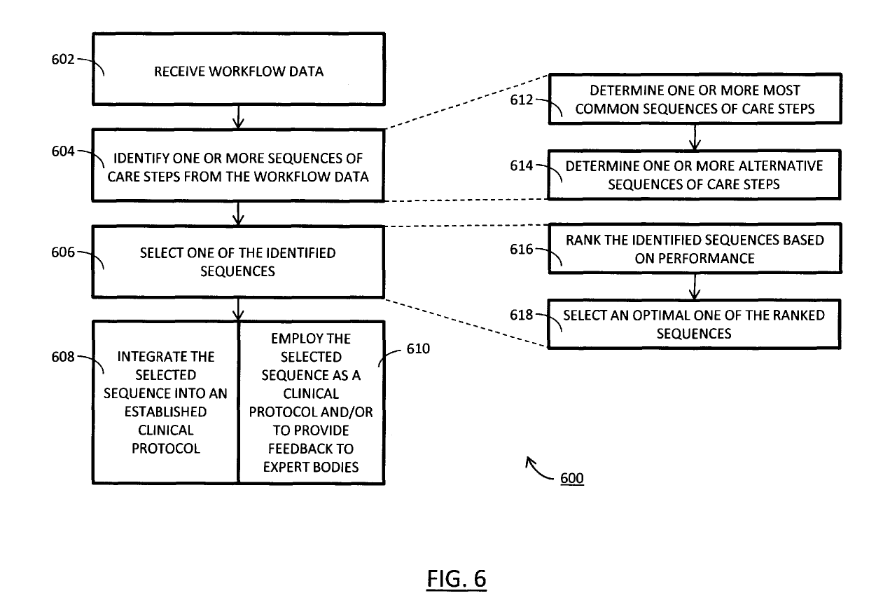

FIG. 6 is a block diagram of a method of learning and/or optimizing clinical protocols and/or clinical guidelines.

With reference to FIG. 1, a block diagram of an information technology (IT) infrastructure 100 of a medical institution, such as a hospital, is provided. The IT infrastructure 100 typically includes one or more clinical devices 102, a communications network 104, a patient information system 106, one or more auxiliary systems 108, a guideline-based clinical decision support system (CDSS) 110, and the like. However, it is to be understood that more or less components and/or different arrangements of components are contemplated.

The clinical devices 102 include one or more of patient monitors, devices at patient beds, mobile communications devices carried by clinicians, clinician workstations, and the like at various physical locations in the medical institution. Further, each of the clinical devices 102 is associated with one or more patients and/or one or more clinicians. For example, a patient monitor attached to a patient and/or a clinician's workstation configured to receive clinical recommendations for a plurality of patients. Each of the patients associated with the clinical devices 102 is associated with one or more clinical problems, such as diseases or medical conditions.

As illustrated, the clinical devices 102 include a patient monitor 102a, a therapeutic device 102b, and a medical imaging device 102c. Communications units 112, 114, 116 of the clinical devices 102 facilitate communication with external systems and/or databases, such as the guideline-based CDSS 110, via the communications network 104. Memories 118, 120, 122 of the clinical devices 102 store executable instructions for performing one of more of the functions associated with the clinical devices 102. Displays 124, 126, 128 of the clinical devices 102 allow the clinical devices 102 to display data and/or messages for the benefit of corresponding users. User input devices 130, 132, 134 of the clinical devices 102 allow the corresponding users of the clinical devices 102 to interact with the clinical devices 102 and/or respond to messages displayed on the displays 124, 126, 128. Controllers 136, 138, 140 of the clinical devices 102 execute instructions stored on the memories 118, 120, 122 to carry out the functions associated with the clinical devices 102.

The communications network 104 allows communication between components of the medical institution connected thereto, such as the guideline-based CDSS 110 and the clinical devices 102, and is suitable for the transfer of digital data between the components. Suitably, the communications network 104 is a local area network. However, it is contemplated that the communications network 104 is one or more of the

Internet, a wide area network, a wireless network, a wired network, a cellular network, a data bus, such as USB and I2C, and the like.

The patient information system 106 acts as a central repository of electronic medical records (EMRs) for patients. Patient data from the clinical devices 102 and other devices generating patient data are suitably stored in the patient information system 106.

In some instances, patient data are received directly from the source of the patient data, and, in other instances, patient data are received indirectly from the source of the patient data. For example, patient data generated by one of the clinical devices 102 are received indirectly from the guideline-based CDSS 110.

Typically, the patient information system 106 includes one of more of a database 142, a server 144, and the like. The database 142 stores EMRs for patients of the medical institution. The server 144 allows components of the medical institution to access to the EMRs via the communications network 104. A communications unit of the server 144 facilitates communication between the server 144 and external devices, such as the clinical devices 102, via the communications network 104. The communications unit 146 further facilitates communication with the database 142 of the patient information system 106. A memory 148 of the server 144 stores executable instructions for performing one of more of the functions associated with the server 144. A controller 150 of the server 144 executes instructions stored on the memory 148 to carry out the functions associated with the server 144.

The auxiliary systems 108 act as central repositories of data relevant to evaluating the performance of care steps performed by clinicians from the perspective of one or more stakeholders, such as the medical institution, administrators, clinicians, patients, and the like. For example, it is contemplated that the data includes administrative data, such as billing data and scheduling data. Billing data is relevant in the sense that it provides the monetary cost of care steps. Scheduling data is relevant in the sense that it provides the effect of care steps on resource utilization. In certain embodiments, the auxiliary systems 108 include one or more of a billing system 108a, a scheduling system, and the like. Further, it is contemplated that each of the auxiliary systems 108 includes a similar configuration as the billing system 108a, described below.

As illustrated, in one embodiment, the billing system 108a includes a database 152 and a server 154. The database 152 stores billing data and the server 154 provide components of the medical institution, such as the guideline-based CDSS 110, with access to the billing data via the communications network 104. A communications unit 156 of the billing system 108a facilitates communication with external systems, such as the guideline-based CDSS 110, via the communications network 104. The communications unit 156 further facilitates communication with the database 152 of the billing system 108a. A memory 158 of the billing system 108a stores executable instructions for performing one of more of the functions associated with the billing system 108a. A controller 160 of the billing system 108a executes instructions stored on the memory 158 to carry out the functions associated with the billing system 108a.

The guideline-based CDSS 110 receives patient data from one or more clinical data sources 162 (see FIG. 2) and, in certain embodiments, provides clinical recommendations based on clinical protocols and/or clinical guidelines to one or more consuming clinical applications 164 (see FIG. 2) to promote adherence to the clinical protocols and/or clinical guidelines.

The clinical data sources 162 provide patient data for associated patients to the guideline-based CDSS 110. The patient data suitably includes clinical data, such as patient symptoms (e.g., chief complaints), patient findings (e.g., physical exam findings), laboratory data (e.g., creatinine values), physiological data (e.g., blood pressure), workflow data, identification data (e.g., patient IDs), and the like. It is contemplated that the workflow data identifies, for example, one or more of care steps performed, care steps currently being performed, care steps yet to be performed, and the like. Suitably, clinical data sources 162 continuously provide patient data to the guideline-based CDSS 110 as it becomes available. For example, if a patient monitor collects data from associated sensors every 5 seconds, newly collected patient data is provided every 5 seconds. However, it is contemplated that events other than the availability of patient data, such as timer events, workflow events, user input events, and the like result in the provisioning of patient data.

The consuming clinical applications 164 receive clinical recommendations for associated patients from the guideline-based CDSS 110. The clinical recommendations specify how to care for the associated patients, so as to assist clinicians with the treatment of the associated patients. To receive clinical recommendations for a patient, a consuming clinical application suitably registers with the guideline-based CDSS 110 to receive clinical recommendations for the patient.

The clinical data sources 162 suitably include at least one of: (1) one or more of the clinical devices 102; (2) the patient information system 106; (3) one or more of the auxiliary systems 108; (4) other devices and/or applications generating patient data; (5) the CDSS 110, such as a user input device thereof; and (6) the like. The consuming clinical applications 164 suitably include at least one of: (1) one or more of the clinical devices 102; (2) the patient information system 106; (3) one or more of the auxiliary systems 108; (4) applications running on devices (e.g., PCs, cell-phones, etc.); (5) the CDSS 110; and (6) the like. In certain embodiments, one or more of the components of the IT infrastructure 100 belong to both the clinical data sources 162 and the consuming clinical applications 164.

The guideline-based CDSS 110 further facilitates learning and/or optimization of clinical protocols and/or clinical guidelines using the received patient data. Although discussed below, briefly, it accomplishes this by providing clinicians with tools to document workflow data and see the most common sequences of care steps employed when treating a clinical problem, such as a disease or a condition. Even more, it provides clinicians with tools to optimize clinical protocols and/or clinical guidelines through performance of a cost benefit analysis in which the considerations of multiple stakeholders are taken in to account.

It is contemplated that the guideline-based CDSS 110 includes one or more devices, servers, computers, database, and the like implementing varying functional aspects of the guideline-based CDSS 110, described in detail hereafter. Further, although described as part of the guideline-based CDSS 110, it is contemplated that the learning and/or optimization of clinical protocols and/or clinical guidelines is performed by a component other than the guideline-based CDSS 110.

With reference to FIG. 2, a detailed view of the functional components of the guideline-based CDSS 110 according to aspects of the present disclosure is provided. The guideline-based CDSS 110 suitably includes a CIG database 166, an instances database 168, a workflow database 170, a guideline execution engine 172, a data collection engine 174, a data analysis engine 176, and the like. It is to be appreciated that these functional components are merely abstractions to simplify the discussion hereafter and are not intended to be construed as limiting the structural layout of the guideline-based CDSS 110. For example, it is contemplated that a plurality of the guideline execution engine 172, the data collection engine 174, and the data analysis engine 176 are embodied by the same structural component.

The guideline execution engine 172 executes CIGs embodying clinical protocols of the medical institution. A clinical protocol typically includes one or more ideal care steps and timing for an occurrence of the care steps as a function of patient information and clinical problem. Further, a clinical protocol typically includes recommendations for each care step. It is contemplated that the clinical protocols are derived from clinical guidelines, but other approaches to deriving the clinical protocols are contemplated, such as the approached carried out by the data analysis engine 176 and the data collection engine 174, discussed below. Suitably, the CIGs are stored within the CIG database 166 and indexed by clinical problem. However, it is contemplated that the CIGs are stored in other components of the medical institution.

To execute CIGs, the guideline execution engine 172 creates instances of the CIGs stored in the CIG database 166 relevant to the clinical problems associated with the patients serviced by the medical institution. For example, when a patient with a particular clinical problem is admitted to the medical institution, the guideline-based CDSS 110 locates one or more ones of the CIGs in the CIG database 166 relevant to the patient and creates instances of the CIGs for the patient. An instance of a CIG is a copy of a CIG tailored to a particular patient with patient data. The instances are suitably maintained in the instances database 168 and indexed by patient. However, it is contemplated that the instances are stored in other components of the medical institution.

The guideline execution engine 172 further maintains and/or updates the instances of the CIGs. As patient data relevant to one of the instances becomes available, the instance is updated to reflect the updated patient information. For example, as a care step is performed for a particular patient, it is contemplated that an associated instance is updated to reflect that the care step has been performed. Relevant patient data includes one or more of physiological data, workflow data, and the like. It is contemplated that the patient data is received from the clinical data sources 162. Additionally or alternatively, insofar as the CDSS 110 includes a computer with a display and a user input device, such as a keyboard and/or mouse, it is contemplated that patient data is received directly from clinicians via a graphical user interface.

While the guideline execution engine 172 is executing the CIGs, the guideline execution engine 172 provides clinical recommendations based on the instances of the CIGs to the consuming clinical applications 164 and/or other components of the medical institution. As noted above, a CIG typically includes recommendations for care steps forming it. Hence, as an instance of a CIG is updated by, for example, completing a care step, recommendations from the new care step of the instance are provided to relevant one or more ones of the consuming clinical applications 164. In certain embodiments, the relevant consuming medical devices are the consuming medical devices that registered with the guideline-based CDSS 110 to receive clinical recommendations pertaining to a patient.

The data collection engine 174 collects workflow data pertaining to sequences of care steps actually employed by clinicians for managing a clinical problem, such as a disease or condition. This workflow data includes one or more of what care steps were performed, when they were performed, who performed them, what the cost of performing them was, what the result of performing them was, and the like. The workflow data is suitably stored in a workflow database 170. However, it is contemplated that the workflow data is stored in other components of the medical institution.

The workflow data is suitably collected electronically, but other approaches to collecting the workflow data are contemplated. For example, in certain embodiments, the workflow data is collected manually from clinicians, for example, in a written form. Even more, the workflow data is suitably collected in real time, retrospectively, or prospectively and for a sufficiently long period of time. In certain embodiments, a sufficiently long period of time is a predetermined amount of time, such as 6 months, or a period of time sufficient to collect a predetermined amount of data, such as 100 sequences of care steps for the clinical problem.

In certain embodiments, the workflow data is collected from the clinical data sources 162 and/or other components of the medical institution. As described below, such workflow data is best suited for the generation of clinical protocols since the data is localized to the medical institution. In other embodiments, the workflow data is collected from one or more external sources 178, such as another medical institution 178a and/or a national registry 178b via, for example, a communications network 180, as shown in FIG. 3. In contrast with the workflow data collected locally, this workflow data is not localized. As such, it is best suited for the generation of clinical guidelines, not clinical protocols.

The data analysis engine 176 analyzes the workflow data in the workflow database 170 to facilitate the generation of one or more clinical protocols and/or one or more clinical guidelines. Suitably, this includes identifying one or more most common sequences of care steps for the clinical problem associated with the collected workflow data. Although this is preferably automated, it is contemplated that a physician provides the data analysis engine 176 with user input to facilitate the identification of the most common sequences of care steps. To illustrate, attention is directed to FIG. 4 in which workflow data for a clinical problem is provided. The workflow data includes care steps A-D and the relations therebetween for three patients. The care steps A-D are common to the three patients, but each of the relations between the care steps A-D corresponds to one of the three patients. A sequence of care steps for the first patient and the second patient is A->C->D->B and a sequence of care steps for the third patient is A->C->B->D. Therefore, the most common sequence of care steps from the workflow data of FIG. 4 is A->C->D->B.

In certain embodiments, the most common sequences of care steps include decision points. For example, suppose two sequences of care steps, such as A->B->C and A->B->D, appear equally within the workflow data. Further, suppose all the patients associated with the first sequence of care steps include a particular attribute and all the patients associated with the second sequence of care steps include a different particular attribute. In such a situation, it is contemplated that the most common sequence of care steps includes A, B, C, and D, where a decision point at B determines whether the care step after B is C or D. Additionally or alternatively, in certain embodiments, the sequences of care steps within the workflow data are grouped and a most common sequence of care steps is identified from each of these groups to define the most common sequences of care steps. It is contemplated that grouping is performed for clinically valid reasons explaining variances in the sequences of care steps, such as particular attributes of the patients associated with the sequences of care steps, time of day, and the like.

While it is contemplated that the most common sequences of care steps can be employed to generate clinical protocols and/or clinical guidelines, in certain situations the most common sequences of care steps for a given disease or condition are not necessarily the most optimum. As such, in certain embodiments, the data analysis engine 176 further identifies one or more alternative sequences of care steps and ranks them with the most common sequences of care steps to identify one or more optimal sequences of care steps. Insofar as the most common sequences of care steps include a plurality of sequences of care steps due to, for example, groups, the groups are maintained while generating the alternative sequences of care steps and identifying the optimal sequences of care steps. For example, alternative sequences of care steps and optimal sequences of care steps are identified for each group.

To identify alternative sequences of care steps, in some embodiments, it is contemplated that the care steps in the workflow data and the relations therebetween are combined into a graph. Thereafter, a graph traversal algorithm, such as A*, breadth first, depth first, and the like, is employed to identify the best sequences of care steps using an evaluation function. Suitably, the evaluation function provides a performance score for one of a care step and a path within the graph. It is contemplated that the performance score is determined through a weighing of interests, such as cost, outcome, readmission rate, and the like, of one or more stakeholders, such as patients, clinicians, medical institutions, and the like. Cost and/or outcome are suitably determined from data of one or more ones of the auxiliary systems 108, such as a billing system or a scheduling system. Notably, these auxiliary systems belong to the clinical data sources 162. In certain embodiments, the graph traversal algorithm further employs rules to limit the search space. For example, a rule that prohibits the graph traversal algorithm from exploring a path in which care step B follows care step A. As another example, a rule limiting the amount time the graph traversal algorithm employs searching the graph.

Contemporaneous with or subsequent to the identification of the alternative sequences of care steps, the alternative sequences of care steps are ranked based on performance. It is contemplated that the evaluation function, for example, is employed to evaluate the performance of the alternative sequences of care steps, regardless of whether the graph traversal algorithm is employed to identify the alternative sequences of care steps. Further, the most common sequences of care steps are similarly evaluated based on performance and ranked with the alternative sequences of care steps. In certain embodiments, the performance of the most common sequences of care steps is weighted to give the most common sequences of care steps higher rankings than they would otherwise have.

Typically, to identify the optimal sequences of care steps, the optimal sequences of care steps are manually selected by, for example, clinical specialists of the medical institution. However, it is contemplated that the mostly highly ranked sequences of care steps are automatically selected. In certain embodiments, a predetermined number of the most highly ranked sequences of care steps are presented to key stakeholders, such as clinical specialists of the medical institution. Although typically factored into the rankings via performance, the optimal sequences of care steps are suitably selected through consideration of the ranked sequences of care steps from both clinical and business perspectives. For example, in certain instances, this involves conducting healthcare quality studies on the ranked sequences of care steps under consideration, where multiple ranked sequences of care steps are considered in parallel by different teams (and/or medical institutions).

After identifying the optimal sequences of care steps or, in certain embodiments, the most common sequences of care steps, it is contemplated that they are employed as clinical protocols and/or to generate clinical guidelines. Whether the identified sequences of care steps are employed as clinical protocols or used to generate clinical guidelines depends upon the source of the workflow data, as discussed below. The clinical protocols and/or clinical guidelines are suitably employed within the medical institution, but it is contemplated that they are provided to other medical institutions.

Insofar as the workflow data is collected from within the medical institution, in certain embodiments, the identified sequences of care steps (i.e., the optimal sequences of care steps or the most common sequences of care steps) are employed as clinical protocols within the medical institution. Because the identified sequences of care steps were generated from local workflow data, the identified sequences of care steps are typically complete enough to be employed as clinical protocols. However, further refinement by domain experts is contemplated. Further, in certain embodiments, the optimal sequences of care steps are converted to CIGs for execution by the guideline-based CDSS 110.

The data analysis engine 176 further, in certain embodiments, analyzes the workflow data in the workflow database 170 to facilitate the updating of clinical protocols and/or clinical guidelines based on the workflow data. As described below, this can take a variety of forms.

In one form, this includes adding one or more free floating nodes to workflow graphs representing partial protocols. A free floating node represents a care step that does not have a clear fit within an incomplete clinical protocol. For example, suppose an established clinical protocol includes the care steps of A-D for treating hypoglycemia and collected workflow data shows that a further care step of E is commonly performed when treating hypoglycemia. A knowledge engineer would add care step E to a workflow graph representing the established clinical protocol as a free floating node. Workflow data about the usage of the free floating nodes (e.g., when and by whom the care steps gets completed) is then analyzed in conjunction with workflow data about the usage of all other nodes to determine the most common and/or optimal locations of the free floating nodes within the incomplete clinical protocol. In certain embodiments, the free floating nodes are then automatically incorporated into the established clinical protocol at the most common and/or optimal locations. In other embodiments, the free floating nodes are then presented to a knowledge engineer with suggested locations based on the most common and/or optimal locations, thereby leaving it to the knowledge engineer to add the free floating nodes.

In another form, one or more most common sequences of care steps and/or one or more optimal sequences of care steps are identified as described above. These most common sequences of care steps and/or optimal sequences of care steps are then compared against an established clinical protocol and/or clinical guideline to identify one or more differences. After identifying the differences, the differences are analyzed to determine which of the differences occur most commonly and/or optimize (i.e., improve performance of) the established clinical protocol and/or clinical guideline. In certain embodiments, the differences which occur most commonly and/or optimize the established clinical protocol and/or clinical guideline are then automatically incorporated into the established clinical protocol and/or clinical guideline. In other embodiments, the differences which occur most commonly and/or optimize the established clinical protocol and/or clinical guideline are presented to a knowledge engineer, thereby leaving it to the knowledge engineer to add the differences.

With reference to FIG. 5, a structural view of the guideline-based CDSS 110 is provided. It is contemplated that the guideline-based CDSS 110 is software running on one or more servers, computers, database, and the like, implementing varying functional aspects of the guideline-based CDSS 110. As illustrated, a server 182 of the guideline-based CDSS 110 suitably includes the guideline execution engine 172 and the data collection engine 174. In certain embodiments, each of the guideline execution engine 172 and the data collection engine 174 is embodied by a non-transient computer readable medium having computer executable instructions for performing the tasks associated with the guideline execution engine 172 and/or the data collection engine 174. A communications unit 184 of the server 182 facilitates communication between the server 182 and external devices, such as the clinical devices 102. The communications unit 184 further facilitates communication with the databases 166, 168, 170 of the guideline-based CDSS 110. A memory 186 of the server 182 stores executable instructions for performing one of more of the functions associated with the server 182. In certain embodiments, these instructions include instructions for performing the tasks associated with the guideline execution engine 172 and/or the data collection engine 174. A controller 188 of the server 182 executes instructions of the memory 186, the guideline execution engine 172, or the data collection engine 174.

A computer 190 of the guideline-based CDSS 110 suitably includes the data analysis engine 176. In certain embodiments, the data analysis engine 176 is embodied by a non-transient computer readable medium having computer executable instructions for performing the tasks associated with the data analysis engine 176. A communications unit 192 of the computer 190 facilitates communication between the computer 190 and external devices, such as the clinical devices 102. The communications unit 192 further facilitates communication with the databases 166, 168, 170 of the guideline-based CDSS 110. A memory 194 of the computer 190 stores executable instructions for performing one of more of the functions associated with the computer 190. In certain embodiments, these instructions include instructions for performing the tasks associated with the data analysis engine 176. A display 196 of the computer 190 allows the computer 190 to display a user interface allowing a user, such as a knowledge engineer, to interact with data analysis engine 176. A user input device 198 of the computer 190 allows the user to interact with the user interface. A controller 200 of the computer 190 executes instructions of the memory 194 or the data analysis engine 176.

With reference to FIG. 6, a block diagram of a method 600 of learning and/or optimizing clinical protocols and/or clinical guidelines is provided. Workflow data is received 602 for a plurality of patients. The workflow data includes a plurality of care steps and relations therebetween for each of the patients. One or more sequences of care steps are identified 604 from the workflow data. The identification includes determining 612 one or more most common sequences of care steps from the workflow data. The identified sequences of care steps include the most common sequences of care steps. In certain embodiments, the identification 604 further includes determining 614 one or more alternative sequences of care steps from the workflow data. In such embodiments, the identified sequences of care steps include the alternative sequences of care steps. One of the identified sequences of care steps is then selected 606. In certain embodiments, the selection 606 includes ranking 616 the identified sequences of care steps based on performance and selecting 618 an optimal one of the ranked sequences of care steps. The selected sequence of care steps is integrated 608 into an established clinical protocol and/or employed 610 as a clinical protocol and/or to provide feedback to expert bodies that generate clinical guidelines.

Each of the databases described herein, such as the CIG database 162, suitably include a computer database, where the computer database is embodied by a single computer, distributed across a plurality of computers, or the like. Further, each of the databases suitably stores data in a structured manner facilitating recall and access to such data. Further, as used herein, a memory includes one or more of a non-transient computer readable medium; a magnetic disk or other magnetic storage medium; an optical disk or other optical storage medium; a random access memory (RAM), read-only memory (ROM), or other electronic memory device or chip or set of operatively interconnected chips; an Internet server from which the stored instructions may be retrieved via the Internet or a local area network; or so forth. Further, as used herein, a controller includes one or more of a microprocessor, a microcontroller, a graphic processing unit (GPU), an application-specific integrated circuit (ASIC), a field-programmable gate array (FPGA), and the like; a communications network includes one or more of the Internet, a local area network, a wide area network, a wireless network, a wired network, a cellular network, a data bus, such as USB and I2C, and the like; a user input device includes one or more of a mouse, a keyboard, a touch screen display, one or more buttons, one or more switches, one or more toggles, and the like; and a display includes one or more of a LCD display, an LED display, a plasma display, a projection display, a touch screen display, and the like.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A method of learning and/or optimizing clinical protocols and/or clinical guidelines, said method comprising:
receiving workflow data for a plurality of patients, wherein the workflow data includes a plurality of care steps and relations there between for each of the patients;
identifying one or more sequences of care steps from the workflow data, wherein the identification includes determining one or more most common sequences of care steps from the workflow data, wherein the identified sequences of care steps include the most common sequences of care steps;
selecting one of the identified sequences of care steps; and,
integrating the selected sequences of care steps into an established clinical protocol and/or employing the selected sequences of care steps as a clinical protocol and/or to provide feedback to expert bodies that generate clinical guidelines.

2. The method according to claim 1, wherein the determination of the most common sequences of care steps includes:
grouping sequences of care steps of the workflow data based on clinically valid reasons explaining variances in the sequences of care steps, wherein the most common sequences of care steps include a most common sequence of care steps for each group.

3. The method according to claim 1, wherein the identification of the sequences of care steps further includes determining one or more alternative sequences of care steps from the workflow data, wherein the identified sequences of care steps include the alternative sequences of care steps.

4. The method according to claim 3, wherein the selection of the identified sequences of care steps includes:
ranking the identified sequences of care steps based on performance; and,
selecting an optimal one of the ranked sequences of care steps.

5. The method according claim 4, wherein performance of the identified sequences of care steps is based on competing interests of a plurality of stakeholders, including a plurality of therapeutic results, workflow efficiency, cost, patient satisfaction and scheduling.

6. The method according to claim 1, wherein the workflow data is received from a plurality of medical institutions and/or national registries.

7. The method according to claim 6, wherein the updating includes:
adding one or more free floating nodes to workflow graphs representing the established clinical protocol and/or clinical guideline;
identifying an optimal location and/or most common location for each of the free floating nodes based on the workflow data; and,
adding one or more of the free floating nodes to its optimal location and/or most common location within the established clinical protocol and/or clinical guideline.

8. The method according to claim 6, wherein the updating includes:
comparing the identified sequences of care steps to the established clinical protocol and/or clinical guideline;
identifying one or more differences between the established clinical protocol and/or clinical guideline and each of the identified sequences of care steps from the comparison;
determining which of the differences occur most frequently and/or improve performance of the established clinical protocol and/or clinical guideline;
adding one or more of the differences which occur most frequently and/or improve performance of the established clinical protocol and/or clinical guideline to the established clinical protocol and/or clinical guideline.

9. One or more user input devices and/or one or more processors preprogrammed and/or devices to perform the method according to claim 1.

10. A computer readable medium carrying software which controls one or more processors to perform the method according to claim 1.

11. A system for learning and/or optimizing clinical protocols and/or clinical guidelines, comprising:
a data collection engine that receives workflow data for a plurality of patients, wherein the workflow data includes a plurality of care steps and relations there between for each of the patients;
a data analysis engine that:
identifies one or more sequences of care steps from the workflow data, the identification includes determining one or more most common sequences of care steps from the workflow data, wherein the identified sequences of care steps include the most common sequences of care steps;
select one or more of the identified sequences of care steps; and,
integrate the selected sequence of care steps into an established clinical protocol and/or employ the selected sequence of care steps as a clinical protocol and/or to provide feedback to expert bodies that generate clinical guidelines.

12. The system according to claim 11, wherein the data analysis engine identifies one or more of the sequences of care steps by:
determining one or more alternative sequences of care steps from the workflow data, wherein the identified sequences of care steps include the alternative sequences of care steps.

13. The system according to claim 11, wherein the data analysis engine selects one or more of the identified sequences of care steps by:
ranking the identified sequences of care steps based on performance; and,
selecting an optimal one of the ranked sequences of care steps.

14. (canceled)

15. (canceled)

16. The system according to claim 11, wherein the data collection engine and the data analysis engine are embodied by a common structural component.

17. (canceled)

18. (canceled)

19. (canceled)

20. (canceled)

21. The medical system according to claim 20, further including:
one or more auxiliary systems that include data for evaluating performance of the plurality of care steps, wherein performance of the identified sequences of care steps is partially based on one or more of billing data, scheduling data, and the like.
